(19) United States
(12) Patent Application Publication
(10) Pub. No.: US 2019/0246940 A1
(43) Pub. Date: Aug. 15, 2019

(54) External RF Coil for High Resolution Magnetic Resonance Imaging of the Prostate

(71) Applicant: BOARD OF REGENTS, THE UNIVERSITY OF TEXAS SYSTEM, Austin, TX (US)

(72) Inventors: Robert E. Lenkinski, Dallas, TX (US); Alexander Ivanishev, Dallas, TX (US); Keith M. Hulsey, Dallas, TX (US); Ivan Pedrosa, Dallas, TX (US); Neil Rofsky, Dallas, TX (US); Daniel Costa, Dallas, TX (US)

(21) Appl. No.: 16/329,451

(22) PCT Filed: Aug. 30, 2017
(86) PCT No.: PCT/US2017/049380

§ 371 Date: Feb. 28, 2019

Related U.S. Application Data
(60) Provisional application No. 62/381,266, filed on Aug. 30, 2016.

(51) Int. Cl. A61B 5/055; A61B 5/00; A61B 6/08; G01R 33/3415

(52) U.S. Cl. CPC A61B 5/055; A61B 5/4381; A61B 2576/02; G01R 33/3415; A61B 5/6823; A61B 6/08

(57) ABSTRACT

The present invention includes an apparatus and method for external magnetic resonance imaging and spectroscopy of a prostate comprising: one or more radio frequency (RF) coils adapted to externally image the prostate, wherein the one or more RF coils are sized and shaped to fit within the perineum area of a subject allowing the placement of the one or more RF coils on the perineum, wherein the one or more RF coils are positioned perpendicular to the prostate; and a processor connected to the radio frequency (RF) coils adapted to measure at a resonance frequency of a magnetic resonance imaging (MRI) scanner or a spectrophotometer, wherein a signal received by the one or more RF coils is used to generate an image of the prostate during magnetic imaging.

### TECHNICAL FIELD OF THE INVENTION

The present invention relates in general to the field of imaging, and more particularly, to a novel design for an external RF coil for imaging of the prostate.

### BACKGROUND OF THE INVENTION

Without limiting the scope of the invention, its background is described in connection with imaging of the prostate.

U.S. Pat. Nos. 5,476,095 and 5,348,010, issued to Schnall, et al., entitled “Intracavity probe and interface device for MRI imaging and spectroscopy”, teach an insertable pickup probe and interface network for magnetic resonance imaging and spectroscopy for imaging the male prostate. The device comprises an elongated shaft supporting a patient interface balloon at its distal end that contains a RF receiving coil. The interface balloon comprises an inflatable inner balloon enclosed by a flexible outer balloon, and the receiving coil is positioned between the inner and outer balloons and placed intimately adjacent the region of interest by inflating the inner balloon to expand outwardly against the outer balloon.

Another such invention is taught in U.S. Pat. No. 5,307,814, issued to Kressel, et al., and is entitled “Externally moveable intracavity probe for MRI imaging and spectroscopy.” Briefly, these inventors also teach an insertable intracavity probe for use in magnetic resonance imaging of an area of interest in a body cavity, particularly the colon, that has an elongate shaft with a handle at its proximal end and an inflatable balloon structure at its distal end.

Another example is taught in U.S. Patent Publication No. 2015/0177346, filed by Mazurewitz, et al., and is entitled, “Non-invasive prostate coil for MR systems with vertical main field.” The application is said to teach an apparatus for external use that includes an inductively coupled magnetic resonance local prostate radio frequency coil that includes at least two connected electrically conductive loops and an interface device. At least two connected electrically conductive loops are said to be tuned to receive magnetic resonance radio frequency signal components along an axis of a subject disposed in a main magnetic field (B0) orthogonal to the axis of the subject (i.e. an open MRI system having a vertical magnetic field) and generate one or more currents indicative of the received magnetic resonance signal components. Yet another example is taught in U.S. Patent Publication No. 2012/0293174, filed by Taracila, et al., entitled “Method and apparatus for imaging a subject using local surface coils.” Briefly, these inventors teach a Radio Frequency (RF) coil apparatus for receiving a Magnetic Resonance (MR) image adapted to be worn by a subject being scanned. The device includes an anterior portion, a posterior portion, and a transition portion coupled between the anterior and posterior portions, a first RF receive-only saddle coil including a first coil positioned in the anterior portion and a second coil positioned in the anterior portion, the first RF saddle coil configured to be positioned on the anterior and posterior sides of the subject.

### SUMMARY OF THE INVENTION

In one embodiment, the present invention includes an apparatus for externally magnetic resonance imaging and spectroscopy of a prostate comprising: one or more radio frequency (RF) coils adapted to externally image the prostate, wherein the one or more RF coils are sized and shaped to fit within the perineum area of a subject allowing the placement of the one or more RF coils on the perineum, wherein the one or more RF coils are positioned perpendicular to the prostate; and a processor connected to the radio frequency (RF) coils adapted to measure at a resonance frequency of a magnetic resonance imaging (MRI) scanner or a spectrophotometer, wherein a signal received by the one or more RF coils is used to generate an image of the prostate during magnetic imaging. In one aspect, the apparatus does not stimulate peristalsis during or after imaging the prostate. In another aspect, the performance of the apparatus of the present invention is independent of the size of a patient. In another aspect, the performance of the apparatus is similar or equivalent to a performance of an endorectal coil. In another aspect, the apparatus comprises two radio frequency receiving coils that are D-shaped. In another aspect, the apparatus comprises a dual coil array. In another aspect, the apparatus comprises a single radio frequency receiving coil that forms two D-shaped regions, wherein the linear portion of the D-shaped regions are adjacent. In another aspect, an RF signal received by the RF coils is generated by a 1T, 2T or 3T magnetic resonance imaging scanner. In another aspect, the signal-to-noise ratio is reduced by at least 10, 15, 20, 25, or 30%. In another aspect, the quality of the image is at least 30, 40, 50, 60, or 70% of a quality of an image obtained with an endorectal MRI pick-up probe. In another aspect, the impendence of the one or more radio frequency (RF) coils and the MRI are matched. In another aspect, the one or more RF coils are tuned to the Larmour frequency of an MRI scanner is accomplished manually or automatically. In another aspect, the processor optimizes automatically both a tuning and an impedance transformation ratio of the one or more RF coils and the MRI scanner. In another aspect, the RF coil and/or the apparatus is coated and/or encased in an enclosure, wherein the coating or case is RF transparent.

In another embodiment, the present invention includes a method of externally imaging the prostate comprising: positioning one or more radio frequency (RF) coils externally from a subject to image the prostate, wherein the one or more RF coils are sized and shaped to fit within the perineum area of the subject allowing the placement of the one or more RF coils on the perineum, wherein the one or more RF coils are positioned perpendicular to the prostate; and measuring with a processor connected to the radio frequency (RF) coils adapted to measure at a resonance frequency of a magnetic resonance imaging (MRI) scanner or a spectrophotometer, wherein a signal received by the one or more RF coils is used to generate an image of the prostate during magnetic imaging. In one aspect, the apparatus does not stimulate peristalsis during or after imaging the prostate. In another aspect, the performance of the apparatus of the present invention is independent of the size of a patient. In another aspect, the performance of the apparatus is similar or equivalent to a performance of an endorectal coil. In another aspect, the apparatus comprises two radio frequency receiving coils that are D-shaped. In another aspect, the apparatus comprises a dual coil array. In another aspect, the apparatus comprises a single radio frequency receiving coil that forms two D-shaped regions, wherein the linear portion of the D-shaped regions are adjacent. In another aspect, an RF signal is generated by a 1T, 2T or 3T magnetic resonance imaging scanner. In another aspect, the signal-to-noise ratio is reduced by at least 10%. In another aspect, the quality of the image is at least 70% of a quality of an image obtained with an endorectal MRI pick-up probe. In another aspect, the impendence of the one or more radio frequency (RF) coils and the MRI are matched. In another aspect, the one or more RF coils are tuned to the Larmour frequency of an MRI scanner is accomplished manually or automatically. In another aspect, the processor optimizes automatically both a tuning and an impedance transformation ratio of the one or more RF coils and the MRI scanner. In another aspect, the RF coil and/or the apparatus is coated and/or encased in an enclosure, wherein the coating or case is RF transparent.

In another embodiment, the present invention includes an apparatus for magnetic resonance imaging and spectroscopy of a prostate comprising: one or more radio frequency (RF) coils adapted to externally image the prostate, wherein the one or more RF coils are sized and shaped to fit within the perineum area of a subject allowing the placement of the one or more RF coils on the perineum, wherein the one or more RF coils are positioned perpendicular to the prostate; a coating or enclosure that is generally RF transparent around the one or more radio frequency (RF) coils; and a processor connected to the radio frequency (RF) coils adapted to measure at a resonance frequency of a magnetic resonance imaging (MRI) scanner or a spectrophotometer, wherein a signal received by the one or more RF coils is used to generate an image of the prostate during magnetic imaging. In another aspect, the RF coil and/or the apparatus is coated and/or encased in an enclosure.

### BRIEF DESCRIPTION OF THE DRAWINGS

For a more complete understanding of the features and advantages of the present invention, reference is now made to the detailed description of the invention along with the accompanying figures and in which:

FIG. 1 is an image that shows a cross sectional view of the perineum and the prostate for positioning of the external RF coil of the present invention.

FIG. 2 shows a dual coil array for use with the present invention.

FIG. 3 shows a single coil array for use with the present invention.

FIGS. 4A and 4B show phantom images taken using the present invention.

FIG. 5 is an image that shows a T2 weighted axial image take with a figure-8 coil array of the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

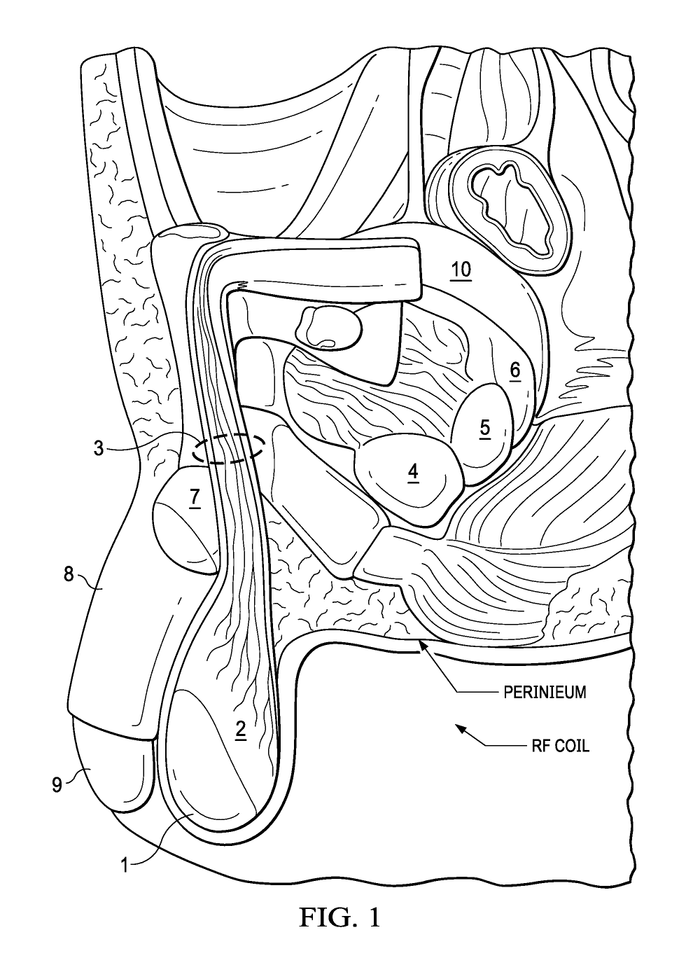

The present invention includes one or more radio frequency (RF) coils that can be placed on the perineum in the position shown in FIG. 1. The user places the RF coil of the present invention in close proximity to the prostate than any existing external coil. Proximity to the organ being imaged determines the quality of the images obtained since the signal-to-noise (SNR) of the images is highly dependent on the distance between the coil and the organ.

Currently, the highest quality MR images (and the most accurate images used for diagnosis of prostate cancer) are obtained at 3T with an intracavitary endorectal coil (U.S. Pat. Nos. 5,348,010 (1994), 5,307,814 (1994), 5,476,095 (1995). This endorectal coil produces about a factor of 3 better SNR than external coil arrays. The RF coil disclosed herein has about 70% of the signal-to-noise (SNR) of the endorectal coil (ERC). Although the ERC performs better than any current alternative, it is inconvenient to use, stimulates peristalsis causing motion artifacts, disrupts the routine clinical MRI workflow, and is not always tolerated by the patient. The RF coil of the present invention incorporates a novel efficient electronic design.

FIG. 1 is a cross sectional view of a male body that shows the relative position of the area to be imaged, namely, the prostate gland 4, in relation to the perineum. The cross sectional view of the body includes the testis 1, the epididymis 2, spermatic cord 3, prostate gland 4, seminal vesicle 5, vas deferens 6, penis 8, penis glans 9 and the urinary bladder 10. The surface of the perineum is indicated. Generally, the surface area of the perineum can be between ½ inch by ½ inch (e.g., an infant), to 1-3 inches by 1-4 inches and is generally defined between the surface of the body starting at the glans to the anus, and between the two legs. Of course, the surface area will vary depending on the age, weight, and height of the individual. However, these variations are almost independent of the size of the patient. The present invention performs in a manner similar or equivalent to that of an insertable endorectal coil, but without the pain, discomfort, and potential damage of inserting a device intrarectally. As such, the performance of the RF coil of the present invention is similar to that of the endorectal coil, is independent of the size of the patient, and does not inconvenience or hurt the patient. The RF coil of the present invention is sized to be used within this area without causing any discomfort. Generally, the RF coil and any coating or enclosure will have an area less than the one described above for the area of the perineum. An arrow shows the location for the use of the RF coil. One advantage of the present invention is that once the signal received at the RF coil, the signal received is processed like the signal received with an endorectal coil, with which skilled artisans are familiar.

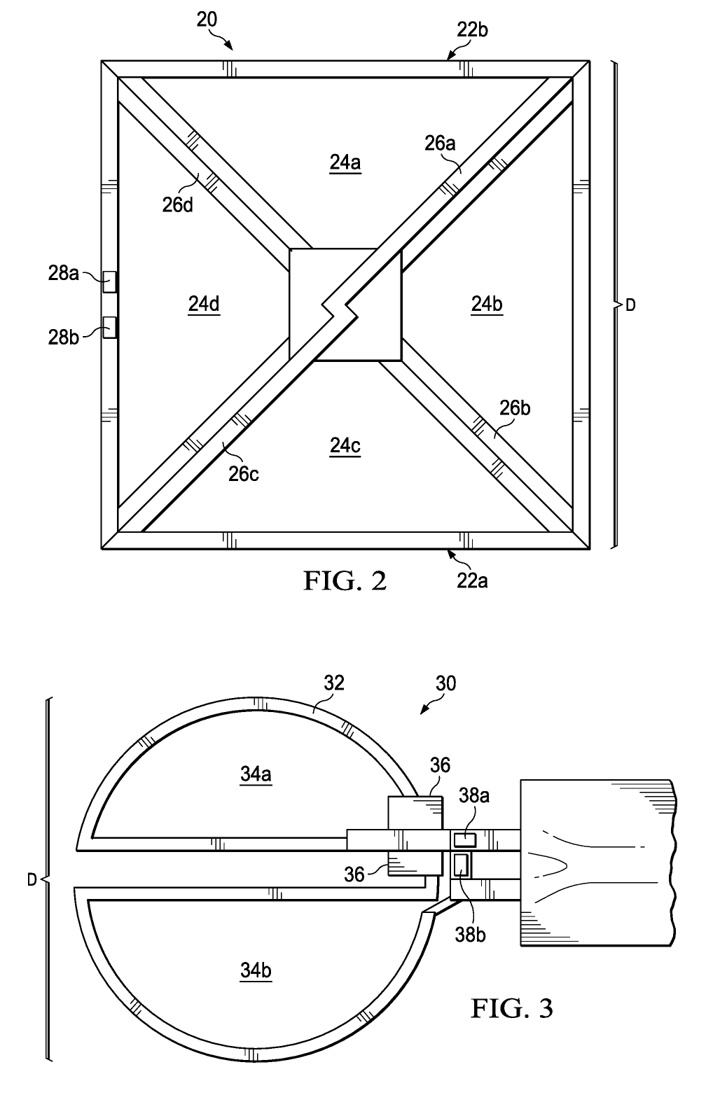

FIG. 2 shows one embodiment of the present invention, a dual coil array 20 is depicted that includes two separate coils 22a, 22b formed into a generally square shape with four generally triangular shapes 24a, 24b, 24c, 24d forming the pair of coils. Dielectrics 26a, 26b, 26c, 26d are shown that provide electrical isolation between the coils 22a, 22b. Electrical contacts 28a, 28b are shown that can be connected to a processor (not depicted) that receives and processes the signals received by the dual coil array 20. The distance D is generally less than a length and/or a width of the perineum.

FIG. 3 shows another embodiment of the present invention, a single coil array 30 is depicted that includes a single coil 32 formed into two D-shaped arrays 34a, 34b. A dielectric or isolators 36 are shown to provide electrical isolation between different parts of the single coil 32. Electrical contacts 38a, 38b are shown that can be connected to a processor (not depicted) that receives and processes the RF signals received by the single coil array 30. The distance D is generally less than a length and/or a width of the perineum.

The RF coil(s) can be made from any conductive material. Non-limiting examples of RF conducting materials include copper, aluminum, silver, and gold, indium oxide, tin oxide, and indium tin oxide (ITO), or alloys thereof. However, other materials may also be used so long as they are RF conductive. The RF conducting material for the RF receiving coils can be coated or encased to protect the RF coils with a polymer, plastic, polypropylene, polystyrene, glass, silica, alumina, oxides of yttria and zirconium, quartz, fiberglass, plexiglass, etc., so long as the material is generally RF transparent. RF transparent materials, or those that cause low RF loss, are those that do not interfere with the RF signals transmitted and received by the RF coils. The RF coil(s) and the protective coating or enclosure can be selected to be reusable or can be single or limited use, including single-use. The protective coating or enclosure can be selected to be sterilizable (one or more times) and/or biocompatible. The RF coil(s) can even be printed onto a substrate, which substrate can form part of an enclosure, or the RF coil(s) and the substrate can be further enclosed. Materials for making an enclosure will generally be biocompatible and RF transparent.

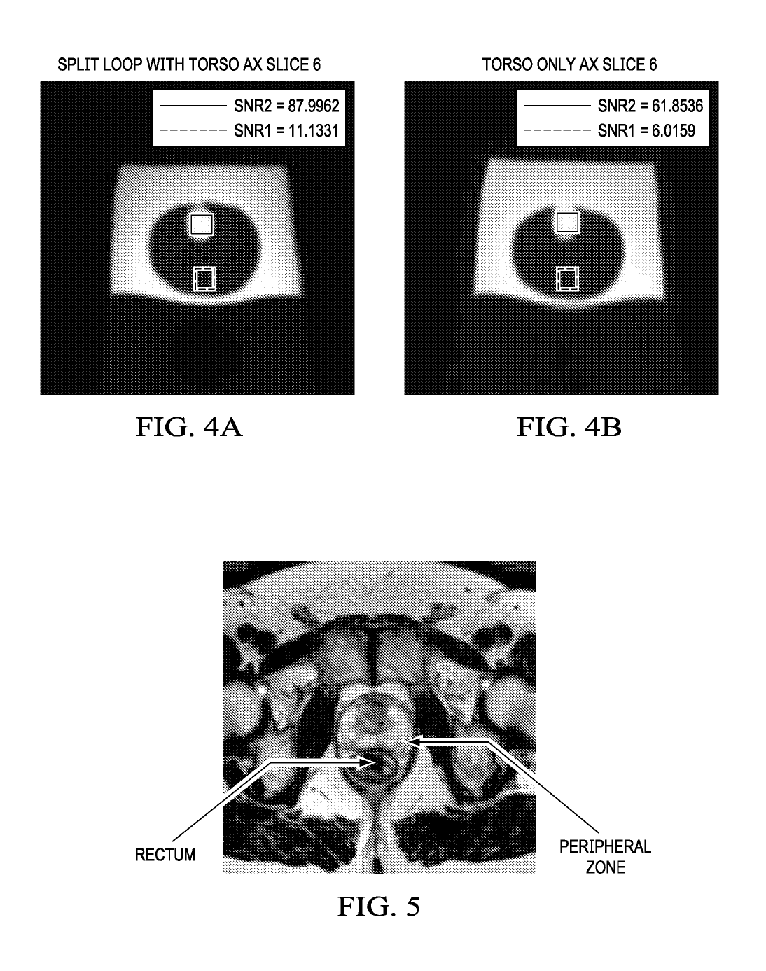

FIGS. 4A and 4B show phantom images used to evaluate the performance of the Figure eight perineal RF coil combined with an external product pelvic array relative to the external array alone on an anthropromorphic prostate phantom (www.yezitronix.com/MultimodalityProstatePhantom.html) on the clinical protocol based on axial T2 weighted scans. An example of the signal-to-noise (SNR) comparison is shown in FIGS. 4A and 4B. There was at least a 40% improvement in the region outlined in the upper box and an 85% improvement in SNR in the region outlined in the lower box. FIGS. 4A and 4B are axial T2 weighted images of the prostate phantom described above. The image in FIG. 4A was acquired with the Figure eight perineal RF coil of the present invention (FIG. 3) combined with an external product pelvic array. The image in FIG. 4B was acquired with the pelvic array alone.

The present inventors imaged four normal volunteers at 3T using the present invention. FIG. 5 is representative of the images obtained. Briefly, the inventors compared the signal-to-noise (SNR) of the MR images obtained with the standard external pelvic array with the SNR of the external figure-eight (figure-8) coil combined with seven of the elements of the pelvic array. The use of the new external coil improved the SNR by approximately 30% in all of these studies. Moreover, as expected from the new design, the SNR advantage was independent of the patient size, which is not the case with the external pelvic array. FIGS. 4A and 4B are examples of a T2 weighted axial image of a slice obtained from one of the volunteers. It is important to note the clear details in the peripheral zone of the prostate. These results are consistent with phantom studies and results of simulations performed on the figure-8 coil. As such, the present invention provides a significant improvement over the insertable endorectal coil of the prior art. The present invention obtains superior results without being inserted into the patient and is independent of the size of the patient. These results are consistent with our phantom studies and results of simulations performed on the FIG. 8 coil shown in FIG. 4A and 4B.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. In certain embodiments, the present invention can be “consisting essentially of” or “consisting of” with regard to the claim elements. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “equivalent”, “similar”, “substantial” or “substantially” refers to the reception of a signal that when so modified is understood to not necessarily be absolute or perfect but would be considered close enough to those of ordinary skill in the art to warrant designating the device as operating in a satisfactory manner. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about”, “equivalent”, or “similar” may vary from the stated value by at least ±1, 2, 3, 4, 5, 6, 7, 10, 12 or 15%. In the specific context of the present invention, the apparatus functions in a manner that is “about”, “equivalent”, or “similar” to an endorectal coil when the device of the present invention images a prostate without being inserted into a patient intrarectally such that the prostate can be imaged and the image provides useful information to the skilled artisan about the prostate.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. An apparatus for external magnetic resonance imaging and spectroscopy of a prostate comprising:
one or more radio frequency (RF) coils adapted to externally image the prostate, wherein the one or more RF coils are sized and shaped to fit within the perineum area of a subject allowing the placement of the one or more RF coils on the perineum, wherein the one or more RF coils are positioned perpendicular to the prostate; and
a processor connected to the radio frequency (RF) coils adapted to measure at a resonance frequency of a magnetic resonance imaging (MM) scanner or a spectrophotometer, wherein a signal received by the one or more RF coils is used to generate an image of the prostate during magnetic imaging.

2. The apparatus of claim 1, wherein the apparatus does not stimulate peristalsis during or after imaging the prostate.

3. The apparatus of claim 1, wherein a performance of the apparatus is independent of the size of a patient, or apparatus is similar or equivalent to a performance of an endorectal coil, or both.

4. (canceled)

5. The apparatus of claim 1, wherein the apparatus comprises two radio frequency receiving coils that are D-shaped, is a dual coil array, or is a single radio frequency receiving coil that forms two D-shaped regions, wherein the linear portion of the D-shaped regions are adjacent.

6. (canceled)

7. (canceled)

8. The apparatus of claim 1, wherein an RF signal is generated by a 1T, 2T or 3T magnetic resonance imaging scanner.

9. The apparatus of claim 1, wherein the signal-to-noise ratio is reduced by at least 10, 15, 20, 25, or 30%, or the quality of the image is at least 30, 40, 50, 60, or 70% of a quality of an image obtained with an endorectal Mill pick-up probe, or both.

10. (canceled)

11. The apparatus of claim 1, wherein the impendence of the one or more radio frequency (RF) coils and the MRI are matched.

12. The apparatus of claim 1, wherein the one or more RF coils are tuned to the Larmour frequency of an MRI scanner is accomplished manually or automatically.

13. The apparatus of claim 1, wherein the processor optimizes automatically both a tuning and an impedance transformation ratio of the one or more RF coils and the MRI scanner.

14. The apparatus of claim 1, wherein the apparatus is coated and/or encased in an enclosure.

15. A method of externally imaging the prostate comprising:
positioning one or more radio frequency (RF) coils externally from a subject to image the prostate, wherein the one or more RF coils are sized and shaped to fit within the perineum area of the subject allowing the placement of the one or more RF coils on the perineum, wherein the one or more RF coils are positioned perpendicular to the prostate; and
measuring with a processor connected to the radio frequency (RF) coils adapted to measure at a resonance frequency of a magnetic resonance imaging (MRI) scanner or a spectrophotometer, wherein a signal received by the one or more RF coils is used to generate an image of the prostate during magnetic imaging.

16. The method of claim 15, wherein the apparatus does not stimulate peristalsis during or after imaging the prostate.

17. The method of claim 15, wherein a performance of the apparatus is independent of the size of a patient, or apparatus is similar or equivalent to a performance of an endorectal coil, or both.

18. (canceled)

19. The method of claim 15, wherein the apparatus comprises two radio frequency receiving coils that are D-shaped, is a dual coil array, or is a single radio frequency receiving coil that forms two D-shaped regions, wherein the linear portion of the D-shaped regions are adjacent.

20. (canceled)

21. (canceled)

22. The method of claim 15, wherein an RF signal is generated by a 1T, 2T or 3T magnetic resonance imaging scanner.

23. The method of claim 15, wherein the signal-to-noise ratio is reduced by at least 10, 15, 20, 25, or 30%, or the quality of the image is at least 30, 40, 50, 60, or 70% of a quality of an image obtained with an endorectal MRI pick-up probe, or both.

24. (canceled)

25. The method of claim 15, wherein the impendence of the one or more radio frequency (RF) coils and the MRI are matched.

26. The method of claim 15, wherein the one or more RF coils are tuned to the Larmour frequency of an MRI scanner is accomplished manually or automatically.

27. The method of claim 15, wherein the processor optimizes automatically both a tuning and an impedance transformation ratio of the one or more RF coils and the MRI scanner.

28. The method of claim 15, wherein the apparatus is coated and/or encased in an enclosure.

29. An apparatus for external magnetic resonance imaging and spectroscopy of a prostate comprising:
one or more radio frequency (RF) coils adapted to externally image the prostate, wherein the one or more RF coils are sized and shaped to fit within the perineum area of a subject allowing the placement of the one or more RF coils on the perineum, wherein the one or more RF coils are positioned perpendicular to the prostate;
a coating or enclosure that is generally RF transparent around the one or more radio frequency (RF) coils; and
a processor connected to the radio frequency (RF) coils adapted to measure at a resonance frequency of a magnetic resonance imaging (MRI) scanner or a spectrophotometer, wherein a signal received by the one or more RF coils is used to generate an image of the prostate during magnetic imaging.
